(19) United States
(12) Patent Application Publication
(10) Pub. No.: US 2020/0046870 A1
(43) Pub. Date: Feb. 13, 2020

(54) MATERIALS COMPRISING HYDROPHOBICALLY-MODIFIED BIOPOLYMER

(71) Applicant: GEL-E LIFE SCIENCES, College Park, MD (US)

(72) Inventors: Matthew DOWLING, College Park, MD (US); Larry TIFFANY, College Park, MD (US)

(21) Appl. No.: 16/342,659

(22) PCT Filed: Oct. 17, 2017
(86) PCT No.: PCT/US2017/056887

§ 371 Date: Apr. 17, 2019

Related U.S. Application Data
(60) Provisional application No. 62/408,923, filed on Oct. 17, 2016.

(51) Int. Cl. A61L 15/28; C08B 37/08; A61L 15/46; A61L 26/00; D06M 15/03; D06M 16/00; D06M 23/04; D01F 9/00

(52) U.S. Cl. CPC A61L 15/28; C08B 37/003; A61L 15/46; A61L 26/0023; A61L 2300/406; D06M 15/03; D06M 16/00; D06M 23/04; D01F 9/00; A61L 26/0066

(57) ABSTRACT

In various aspects, the invention provides materials comprising a hydrophobically-modified biopolymer, such as hm-chitosan, as well as methods of making the materials, including textile materials. The hm-biopolymer can be a polysaccharide and may have antimicrobial properties including against antibiotic-resistant bacteria, providing opportunities to combat microbial persistence in clothing and at wound sites.

### BACKGROUND

There is a need for high performing products that are antimicrobial, odor-resistant, and/or durable, including for specialty applications such as sportswear, undergarments, hospital apparel, and healthcare applications, among others. Such materials should demonstrate high performance in terms of, for example, longevity, durability, comfort, and hygeine.

The present invention addresses these and other objectives.

### SUMMARY

Chitosan is a robust, durable material which can be incorporated into textiles and other materials. The addition of hydrophobic grafts to the backbone of chitosan introduces various properties that are beneficial in the field of textiles, for example, including introduction or enhancement of antibacterial and/or antifungal properties.

In various aspects, the invention provides a textile material comprising a hydrophobically-modified biopolymer, such as hydrophobically-modified (hm) chitosan, as well as methods of making such textile materials. The hm-biopolymer has antimicrobial properties, including against common pathogens (including drug-resistant bacteria) and odor-causing microbes, providing opportunities for creating microbial-resistant and odor-resistant clothing, as well as providing important advantages in developing durable and more hygienic textile materials.

In various embodiments, the material can be engineered for the desired application by selection of biopolymer properties, such as biopolymer molecular weight, amount of available amines or other functional group, type and amount of hydrophobic moieties, and processing technique of the hydrophobically-modified biopolymer for use in the desired textile application. In accordance with embodiments of the invention, textiles can be engineered for a wide range of properties, including antimicrobial activity, odor-resistance, durability, flexibility, feel and comfort, and/or water repellant character.

The hm-biopolymer can be formed into fibers for preparation of textiles, and/or can be combined with various natural and synthetic fibers. In some embodiments, the textile material is prepared from fibers formed from a dehydrated solution or foam of hm-chitosan.

In other aspects, the invention provides methods for making textiles that incorporate hm-biopolymers, such as hm-chitosan. The method in some embodiments comprises incorporating an hm-biopolymer in accordance with this disclosure into one or more natural or synthetic fibers, and preparing a textile material from the resulting material. In other embodiments, the invention comprises preparing textile fibers with a material comprising the hm-biopolymer.

In still other aspects, the anti-microbial properties of hm-chitosan are applied to protect the integrity of skin grafts and surgical wounds, and prevent or treat infection from drug-resistant bacteria.

Other aspects and embodiments will be described in greater detail below.

### DESCRIPTION OF THE FIGURES

FIG. 1 shows the antibacterial activity of hydrophobically-modified chitosan in a bacterial clearing test. 10 μl of 0.5% hydrophobically-modified chitosan solution produce clearing zones up to 10 mm in diameter.

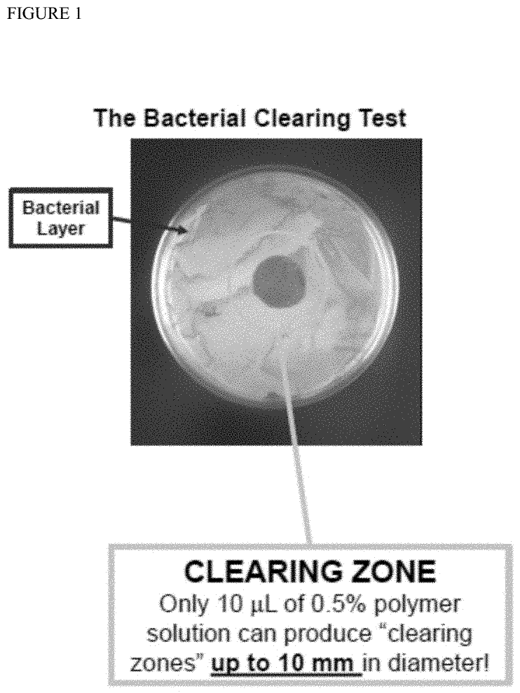

FIG. 2 compares the antimicrobial properties of chitosan and hydrophobically modified chitosan, alongside ampicillin, against Methicillin-resistant Staphylococcus aureus (MRSA). Hm-chitosan at 0.5 wt % achieves a log killing of >2, whereas native chitosan (0.5 wt %) achieves a log killing of ˜1. In contrast, ampicillin at high dose (100 μg/ml) achieves only ˜0.5 log killing.

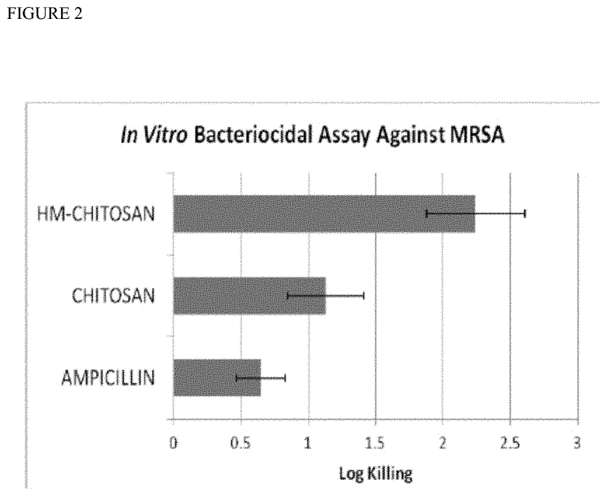

### DETAILED DESCRIPTION

In various aspects, the invention provides a textile material comprising a hydrophobically-modified biopolymer, such as hm-chitosan, as well as methods of making such textile materials. The hm-biopolymer can be a polysaccharide and may have antimicrobial properties, providing opportunities for odor-resistant clothing that is both durable and hygienic.

An exemplary hm-polymer material is hm-chitosan. Chitosan is the common name of the linear, random copolymer that consists of β-(1-4)-linked D-glucosamine and N-acetyl-D-glucosamine. The molecular structure of chitosan consists of a linear backbone linked with glycosidic bonds. Chitosan is the major component of crustacean shells such as crab, shrimp, krill and crawfish shells. Additionally, chitosan is the second most abundant natural biopolymer after cellulose. Commercial chitosan samples are typically prepared by chemical de-N-acetylation of chitin under alkaline conditions. Depending on the source of the natural chitin (extracted from shells) and its production process, chitosan can differ in size (average molecular weight Mw) and degree of N-acetylation (% DA). While the poor solubility of chitosan in water and in common organic solvents restricts its applications, reactive amino groups in the chitosan backbone make it possible to chemically conjugate chitosan with various molecules and to modulate its properties for use in textiles.

The degree of deacetylation of chitin may range from about 40-100%, or in some embodiments, from 60 to 100%, which determines the charge density. The structure of chitosan (deacetylated), and is depicted in Formula 1:

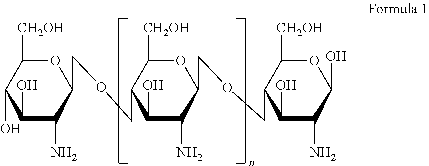

These repeating monomeric units include a free amino group, which makes molecules or compounds containing chitosan or its derivatives readily reactive. The hydrophobic modification of the chitosan backbone is through the association of an amphiphilic compound with the amino group, such that the hydrophobic tail of the amphiphilic compound is bound with the hydrophilic backbone structure.

Without being bound by theory, antimicrobial properties of native chitosan may be based on interactions between protonated amine groups (e.g., NR₃+, where R is H or a substituent), and negatively-charged groups on the microbial cell membranes. Surprisingly, attachment of hydrophobic grafts to the backbone of the chitosan molecule through the available amines can enhance antimicrobial activity against some pathogens, including Methicillin-resistant Staphylococcus aureus (MRSA), a common skin pathogen that can result in dangerous infectious and which is highly contagious. For example, MRSA can live in towels and clothing, and even contaminate washing machines, allowing the dangerous bacteria to spread from person-to-person through laundry. Further, hm-chitosan is a stable, robust, and durable biopolymer which is capable of retaining its functionality for extremely long storage periods at room temperature.

The polymer that forms the backbone is chitosan, or similar polymer of synthetic or natural origin, including for example, water-soluble polysaccharides and water-soluble polypeptides. In some embodiments, the polymer is one or more hm-polysaccharides, including but not limited to cellulosics, chitosans and alginates, all of which are abundant, natural biopolymers. In some embodiments, the hm-biopolymer contains cationic groups.

The natural origin of these polysaccharides varies, cellulosics are found in plants, whereas chitosans and alginates are found in the exoskeleton or outer membrane of a variety of living organisms. Many of these naturally occurring polymers, in addition to being able to form long stable chains for forming the polymer backbone, have properties that are beneficial for textile applications, including anti-microbial properties (a property that can besurprisingly enhanced with hm-chitosan against skin pathogens).

In some embodiments, the hm-chitosan is derived from a deacteylated chitin, which may be derived from one or more of crab, shrimp, krill, and crawfish.

The form of the natural polymers used may vary to include standard states, derivatives and other various formulations. For example, the hm-cellulosics may be formed from, without limitation, hydroxyethyl cellulose, hydroxypropyl cellulose, methyl cellulose, hydroxypropyl methyl cellulose, and/or hydroethyl methyl cellulose. Hm-chitosans may be prepared from, without limitation, the following chitosan salts: chitosan lactate, chitosan salicylate, chitosan pyrrolidone carboxylate, chitosan itaconate, chitosan niacinate, chitosan formate, chitosan acetate, chitosan gallate, chitosan glutamate, chitosan maleate, chitosan aspartate, chitosan glycolate and quaternary amine substituted chitosan and salts thereof. Hm-alginates may be prepared from, without limitation, sodium alginate, potassium alginate, magnesium alginate, calcium alginate, and/or aluminum alginate. It is to be understood that various other forms of any of these natural polysaccharides that provide the proper functional capabilities may be employed without departing from the scope and spirit of the present invention.

In some embodiments, the polymeric component is a mixture of polysaccharides. For instance, the mixture may be of various different sub-classes of a single polymer class. Alternatively, the mixture may include two or more different classes of polymer, for instance a cellolusic and a chitosan.

In various embodiments, the biopolymer is a hm-chitosan, which may be prepared from a chitosan having a degree of deacetylation of from about 40% to about 90%, such as from about 50% to about 80%, such as from about 60% to about 75%. In some embodiments, the degree of substitution of the hydrophobic substituent on the biopolymer is from about 1 to about 100 moles of the hydrophobic substituent per mole of the biopolymer. In some embodiments, the degree of substitution of the hydrophobic substituent on the polysaccharide is from about 40 to 65 moles of the hydrophobic substituent per mole of the polysaccharide. In some embodiments, the degree of substitution of the hydrophobic substituent on the polysaccharide is from about 1 to 30 moles of the hydrophobic substituent per mole of the polysaccharide. In some embodiments, the molecular weight of the polysaccharides used as the biopolymer range from about 25,000 to about 1,500,000 grams per mole. In various embodiments, the molecular weight of the biopolymer ranges from about 40,000 to about 500,000 grams per more, or from about 50,000 to about 250,000 grams per mole, or from about 50,000 to about 100,000 grams per mole. As used herein, the term “molecular weight” means weight average molecular weight. Methods for determining average molecular weight of bio-polymers include low angle laser light scattering (LLS) and Size Exclusion Chromatography (SEC). In performing low angle LLS, a dilute solution of the polysaccharide, typically 2% or less, is placed in the path of a monochromatic laser. Light scattered from the sample hits the detector, which is positioned at a low angle relative to the laser source. Fluctuation in scattered light over time is correlated with the average molecular weight of the polysaccharide in solution. In performing SEC measurements, again a dilute solution of biopolymer, typically 2% or less, is injected into a packed column. The polysaccharide is separated based on the size of the dissolved polymer molecules and compared with a series of standards to derive the molecular weight.

A hydrophobically modified biopolymer material for incorporation into textiles can be based on a solution of the hm-biopolymer that is about 0.1% to about 5.0% by weight relative to the total weight of the solution, or in some embodiments, about 0.5% to about 4%, or about 0.5% to about 3% of the total weight of the solution, or about 0.5% to about 2% of the total weight of the solution. In some embodiments, the solution is about 1.0% to about 5.0% by weight relative to the total weight of the solution of the biopolymer, or in some embodiments, about 1.5% to about 5%, or about 2.0% to about 4% of the total weight of the solution. In some embodiments, the hm-biopolymer solution is dried or lyophilized.

Hydrophobic moieties can be independently selected from saturated hydrocarbons (e.g., alkanes) and unsaturated hydrocarbons (e.g., alkenes, alkynes), which may be linear, branched or cyclic. In some embodiments, the hydrophobic moieties include aromatic hydrocarbons. In some embodiments, the hydrophobic moiety is a hydrocarbon having from about 4 to about 100 carbon atoms, or from about 8 to about 60 carbon atoms, or from about 8 to about 28 carbon atoms, or from about 8 to about 18 carbon atoms.

The hydrophobic substituents may be a hydrocarbon group having from about 8 to about 18 carbon atoms attached to the backbone of the one biopolymer, and in some embodiments comprises an alkyl group. In some embodiments, the hydrocarbon group comprises an arylalkyl group. As used herein, the term “arylalkyl group” means a group containing both aromatic and aliphatic structures.

The textiles may comprise numerous hydrophobically modified biopolymer compounds. These compounds comprise a biopolymer (such as chitosan) backbone that includes a hydrophilically reactive functional group (e.g., amino groups) that binds with the hydrophilically reactive head groups (e.g., carbonyl functional group) of an amphiphilic compound (e.g., aldehyde), to form the hm-chitosan or other hm-polymer. The head group is further associated with a hydrophobic tail group. In the current embodiment, the hydrophobic tail may be, for example, a hydrocarbon. Thus, a hydrophobic tail is associated with the biopolymer backbone providing the hydrophobic modification to the molecule that extends from the backbone and may interact with a surrounding environment in numerous ways, such as through hydrophobic interaction with materials.

Examples of procedures for modifying polymers are as follows.

Alginates can be hydrophobically modified by exchanging their positively charged counterions (e.g. Na+) with tertiary-butyl ammonium (TBA) ions using a sulfonated ion exchange resin. The resulting TBA-alginate is dissolved in dimethylsulfoxide (DMSO) where reaction occurs between alkyl (or aryl) bromides and the carboxylate groups along the alginate backbone. Alginate can also be modified by fatty amine groups (e.g. dodecyl amine), followed by addition of 1-ethyl-3-(3-dimethylaminopropyl)carbodiimide, via EDC coupling.

Cellulosics can be hydrophobically modified by first treating the cellulosic material with a large excess highly basic aqueous solution (e.g. 20 wt % sodium hydroxide in water). The alkali cellulose is then removed from solution and vigorously mixed with an emulsifying solution (for example, oleic acid) containing the reactant, which is an alkyl (or aryl) halide (e.g. dodecyl bromide).

Chitosans can be hydrophobically modified by reaction of alkyl (or aryl) aldehydes with primary amine groups along the chitosan backbone in a 50/50 (v/v) % of aqueous 0.2 M acetic acid and ethanol. After reaction, the resulting Schiff bases, or imine groups, are reduced to stable secondary amines by dropwise addition of the reducing agent sodium cyanoborohydride.

The degree of substitution of the hydrophobic substituent on the polymer is up to 50% of available functional groups, for example, amines in the case of chitosan. For example, the hydrophobic substituent can be added to from 10 to 50% of available amines, or from 20 to 50% of available amine, or from 30 to 50% of available amines. It is contemplated that more than one particular hydrophobic substituent may be substituted onto the polymer, provided that the total substitution level is substantially within the ranges set forth above.

In some embodiments, the hydrophobic substituent is derived from an amphiphilic compound, meaning it is composed of a hydrophilic Head group and a hydrophobic Tail group. The Head group binds with the polymer and positions the Tail group to extend from the backbone of the polymer scaffold. This makes the hydrophobic Tail group available for hydrophobic interactions. The Tail group is a hydrocarbon of various forms.

Hydrocarbons that find use in accordance with this disclosure may be classified as saturated hydrocarbons, unsaturated hydrocarbons, and aromatic hydrocarbons. From this basic classification system there exist many derivatives and further types of compounds that build therefrom. For example, numerous and varied compounds include more than one aromatic ring and are generally referred to as polyaromatic hydrocarbons (PAH). In some embodiments, the hydrophobic moiety is aliphatic. Aliphatic compounds, carbon atoms can be joined together in straight chains, branched chains, or rings (in which case they are called alicyclic). They can be joined by single bonds (alkanes), double bonds (alkenes), or triple bonds (alkynes). Besides hydrogen, other elements can be bound to the carbon chain, the most common being oxygen, nitrogen, sulfur, and chlorine. Those of ordinary skill in the art will recognize that other molecules may also be bound to the carbon chains and that compounds of such heteroatomic structure are contemplated as falling within the scope of the current invention.

The hydrophobic Tail group of the amphiphilic compound bound to the polymer backbone of the current invention is capable of branching and/or allowing the inclusion of side chains onto its carbon backbone. It may be understood that the strength of the hydrophobic interaction is based upon the available amount of “hydrophobes” that may interact amongst themselves or one another. Thus, it may further promote the hydrophobic effect by increasing the amount of and/or hydrophobic nature of the hydrophobic Tail group that is interacting. For instance, a hydrophobic Tail group, which in its original form may include a hydrocarbon chain, may promote an increase in its hydrophobicity (ability to hydrophobically bond and strength of hydrophobic interaction) by having a hydrophobic side chain attach to one of the carbons of its carbon backbone.

In some embodiments, the current invention contemplates the use of various molecules and/or compounds that may increase one or more of antimicrobial activity, durability, water repellent properties, and/or flexibility of the textile material. The side chains may be linear chains, aromatic, aliphatic, cyclic, polycyclic, or any various other types of hydrophobic side chains as contemplated by those skilled in the art.

In some embodiments, the hydrophobic grafts include an alicyclic, cycloalkane, or cycloalkene. For example, the hydrophobic group may be both aliphatic and cyclic with or without side chains attached. In some embodiments, the cyclic groups are carbocyclic groups, which may be saturated or unsaturated (aromatic or non-aromatic).

In some embodiments, the hydrophobic grafts include aromatic hydrocarbon, or polycyclic aromatic hydrocarbon, or heterocyclic moieties. Heterocyclic groups may include, in addition to carbon, at least one atom such as nitrogen, oxygen, or sulfur, as part of the ring. Examples include pyridine (C₅H₅N), Pyrimidine (C₄H₄N₂) and Dioxane.

Some of the contemplated hydrophobic side chains may include the following:

TABLE 1
Linear Alkanes

| Number of | | |
|---|---|---|
| C Atoms | Formula | Common Name |
| 1 | CH₄ | Methane |
| 2 | C₂H₆ | Ethane |
| 3 | C₃H₈ | Propane |
| 4 | C₄H₁₀ | n-Butane |
| 5 | C₅H₁₂ | n-Pentane |
| 6 | C₆H₁₄ | n-Hexane |
| 7 | C₇H₁₆ | n-Heptane |
| 8 | C₈H₁₈ | n-Octane |
| 9 | C₉H₂₀ | n-Nonane |
| 10 | C₁₀H₂₂ | n-Decane |
| 11 | C₁₁H₂₄ | n-Undecane |
| 12 | C₁₂H₂₆ | n-Dodecane |
| 13 | C₁₃H₂₈ | n-Trideacane |
| 14 | C₁₄H₃₀ | n-Tetradecane |
| 15 | C₁₅H₃₂ | n-Pentadecane |
| 16 | C₁₆H₃₄ | n-Hexadecane |
| 17 | C₁₇H₃₆ | n-Heptadecane |
| 18 | C₁₈H₃₈ | n-Octadecane |
| 19 | C₁₉H₄₀ | n-Nonadecane |
| 20 | C₂₀H₄₂ | n-Eicosane |
| 21 | C₂₁H₄₄ | n-Heneicosane |
| 22 | C₂₂H₄₆ | n-Docosane |
| 23 | C₂₃H₄₈ | n-Tricosane |
| 24 | C₂₄H₅₀ | n-Tetracosane |
| 25 | C₂₅H₅₂ | n-Pentacosane |
| 26 | C₂₆H₅₄ | n-Hexacosane |
| 27 | C₂₇H₅₆ | n-Heptacosane |
| 28 | C₂₈H₅₈ | n-Octacosane |
| 29 | C₂₉H₆₀ | n-Nonacosane |
| 30 | C₃₀H₆₂ | n-Triacontane |
| 31 | C₃₁H₆₄ | n-Hentraiacontane |
| 32 | C₃₂H₆₆ | n-Dotriacontane |
| 33 | C₃₃H₆₈ | n-Tritriacontane |
| 34 | C₃₄H₇₀ | n-Tetratriacontane |
| 35 | C₃₅H₇₂ | n-Pentatriacontane |
| 36 | C₃₆H₇₄ | n-Hexatriacontane |
| 37 | C₃₇H₇₆ | n-Heptatriacontane |
| 38 | C₃₈H₇₈ | n-Octatriacontane |
| 39 | C₃₉H₈₀ | n-Nonactriacontane |
| 40 | C₄₀H₈₂ | n-Tetracontane |
| 41 | C₄₁H₈₄ | n-Hentatetracontane |
| 42 | C₄₂H₈₆ | n-Dotetracontane |
| 43 | C₄₃H₈₈ | n-Tritetracontane |
| 44 | C₄₄H₉₀ | n-Tetratetracontane |
| 45 | C₄₅H₉₂ | n-Pentatetracontane |
| 46 | C₄₆H₉₄ | n-Hexatetracontane |
| 47 | C₄₇H₉₆ | n-Heptatetracontane |
| 48 | C₄₈H₉₈ | n-Octatetracontane |
| 49 | C₄₉H₁₀₀ | n-Nonatetracontane |
| 50 | C₅₀H₁₀₂ | n-Pentacontane |
| 51 | C₅₁H₁₀₄ | n-Henpentacontane |
| 52 | C₅₂H₁₀₆ | n-Dopentacontane |
| 53 | C₅₃H₁₀₈ | n-Tripentacontane |
| 54 | C₅₄H₁₁₀ | n-Tetrapentacontane |
| 55 | C₅₅H₁₁₂ | n-Pentapentacontane |
| 56 | C₅₆H₁₁₄ | n-Hexapentacontane |
| 57 | C₅₇H₁₁₆ | n-Heptapentacontane |
| 58 | C₅₈H₁₁₈ | n-Octapentacontane |
| 59 | C₅₉H₁₂₀ | n-Nonapentacontane |
| 60 | C₆₀H₁₂₂ | n-Hexacontane |
| 61 | C₆₁H₁₂₄ | n-Henhexacontane |
| 62 | C₆₂H₁₂₆ | n-Dohexacontane |
| 63 | C₆₃H₁₂₈ | n-Trihexacontane |
| 64 | C₆₄H₁₃₀ | n-Tetrahexacontane |
| 65 | C₆₅H₁₃₂ | n-Pentahexacontane |
| 66 | C₆₆H₁₃₄ | n-Hexahexacontane |
| 67 | C₆₇H₁₃₆ | n-Heptahexacontane |
| 68 | C₆₈H₁₃₈ | n-Octahexacontane |
| 69 | C₆₉H₁₄₀ | n-Nonahexacontane |
| 70 | C₇₀H₁₄₂ | n-Heptacontane |
| 71 | C₇₁H₁₄₄ | n-Henheptacontane |
| 72 | C₇₂H₁₄₆ | n-Doheptacontane |
| 73 | C₇₃H₁₄₈ | n-Triheptacontane |
| 74 | C₇₄H₁₅₀ | n-Tetraheptacontane |
| 75 | C₇₅H₁₅₂ | n-Pentaheptacontane |
| 76 | C₇₆H₁₅₄ | n-Hexaheptacontane |
| 77 | C₇₇H₁₅₆ | n-Heptaheptacontane |
| 78 | C₇₈H₁₅₈ | n-Octaheptacontane |
| 79 | C₇₉H₁₆₀ | n-Nonaheptacontane |
| 80 | C₈₀H₁₆₂ | n-Otcacontane |
| 81 | C₈₁H₁₆₄ | n-Henoctacontane |
| 82 | C₈₂H₁₆₆ | n-Dooctacontane |
| 83 | C₈₃H₁₆₈ | n-Trioctacontane |
| 84 | C₈₄H₁₇₀ | n-Tetraoctacontane |
| 85 | C₈₅H₁₇₂ | n-Pentaoctacontane |
| 86 | C₈₆H₁₇₄ | n-Hexaoctacontane |
| 87 | C₈₇H₁₇₆ | n-Heptaoctacontane |
| 88 | C₈₈H₁₇₈ | n-Octaoctacontane |
| 89 | C₈₉H₁₈₀ | n-Nonaoctacontane |
| 90 | C₉₀H₁₈₂ | n-Nonacontane |
| 91 | C₉₁H₁₈₄ | n-Hennonacontane |
| 92 | C₉₂H₁₈₆ | n-Dononacontane |
| 93 | C₉₃H₁₈₈ | n-Trinonacontane |
| 94 | C₉₄H₁₉₀ | n-Tetranonacontane |
| 95 | C₉₅H₁₉₂ | n-Pentanonacontane |
| 96 | C₉₆H₁₉₄ | n-Hexanonacontane |
| 97 | C₉₇H₁₉₆ | n-Heptanonacontane |
| 98 | C₉₈H₁₉₈ | n-Octanonacontane |
| 99 | C₉₉H₂₀₀ | n-Nonanonacontane |
| 100 | C₁₀₀H₂₀₂ | n-Hectane |
| 101 | C₁₀₁H₂₀₄ | n-Henihectane |
| 102 | C₁₀₂H₂₀₆ | n-Dohectane |
| 103 | C₁₀₃H₂₀₈ | n-Trihectane |
| 104 | C₁₀₄H₂₁₀ | n-Tetrahectane |
| 105 | C₁₀₅H₂₁₂ | n-Pentahectane |
| 106 | C₁₀₆H₂₁₄ | n-Hexahectane |
| 107 | C₁₀₇H₂₁₆ | n-Heptahectane |
| 108 | C₁₀₈H₂₁₈ | n-Octahectane |
| 109 | C₁₀₉H₂₂₀ | n-Nonahectane |
| 110 | C₁₁₀H₂₂₂ | n-Decahectane |
| 111 | C₁₁₁H₂₂₄ | n-Undecahectane |

TABLE 2
Cyclic Compounds

| | | Example |
|---|---|---|
| Polycyclic Compounds | Sub-Types | Compounds |
| Bridged Compound - | Bicyclo compound | adamantine |
| compounds which | | amantadine |
| contain interlocking rings | | biperiden |
| | | memantine |
| | | methenamine |
| | | rimantadine |
| Macrocyclic Compounds | Calixarene | |
| | Crown Compounds | |
| | Cyclodextrins | |
| | Cycloparaffins | |
| | Ethers, Cyclic | |
| | Lactans, macrocyclic | |
| | Macrolides | |
| | Peptides, Cyclic | |
| | Tetrapyrroles | |
| | Trichothecenes | |
| Polycyclic Hydrocarbons, | Acenaphthenes | |
| Aromatic | Anthracenes | |
| | Azulenes | |
| | Benz(a)anthracenes | |
| | Benzocycloheptenes | |
| | Fluorenes | |
| | Indenes | |
| | Naphthalenes | |
| | Phenalenes | |
| | Phenanthrenes | |
| | Pyrenes | |
| | Spiro Compounds | |
| Steroids | Androstanes | |
| | Bile Acids and Salts | |
| | Bufanolides | |
| | Cardanolides | |
| | Cholanes | |
| | Choestanes | |
| | Cyclosteroids | |
| | Estranes | |
| | Gonanes | |
| | Homosteroids | |
| | Hydroxysteroids | |
| | Ketosteroids | |
| | Norsteroids | |
| | Prenanes | |
| | Secsteroids | |
| | Spirostans | |
| | Steroids, Brominated | |
| | Steroids, Chlorinated | |
| | Steroids, Fluorinated | |
| | Steroids, Heterocyclic | |

The hm-modified biopolymer, such as hm-chitosan, can have antimicrobial properties, including antibacterial and/or antifungal properties. In some embodiments, the hm-biopolymer can have antimicrobial properties against one or more common pathogens or odor-causing bacteria or fungus. Examples include: Pseudomonas aeruginosa, Acinetobacter baumanni, Klebsiella pneumonia, Escherichia coli, Staphylococcus aureus and Enterococcus faecalis. In some embodiments, the hm-biopolymer has antimicrobial properties against Methicillin-resistant Staphylococcus aureus (MRSA), a common pathogen found on skin which is easily spread by contact with contaminated surfaces.

In still some embodiments, the hm-biopolymer is active against one or more of Staphylococcus sp., Pseudomonas sp., Enterococcus sp., Shigella sp., Listeria sp., Bacillus sp., Lactobaccillus sp., Salmonella sp., and Vibrio sp. In some embodiments, the hm-polymer has antifungal activity against one or more of Aspergillus sp., Fusarium sp., and Candida sp. The particular biopolymer can be selected in accordance with the disclosure for the desired antibacterial and/or anti-fungal profile, which can depend on the application of the textile. In the case of chitosan, hm-chitosan can have antimicrobial properties greater than native chitosan for certain drug-resistant bacteria, including MRSA. In some embodiments, the hm-polymer is chitosan modified with hydrophobic groups having from 8 to 28 carbon atoms. The hm-polymer can further be designed for the desired durability, flexibility, and/or water repellant nature of the resulting textile, based on, for example, biopolymer molecular weight, amount of available amines or other functional group, type and amount of hydrophobic moieties, and processing technique for the hydrophobically-modified biopolymer for use in textiles. In some embodiments, a foaming agent is incorporated prior to drying to modulate the flexibility and/or feel of the resulting material.

In accordance with embodiments, the hm biopolymer is incorporated into a natural or synthetic fiber, or alternatively, is used for the preparation of fibers, including yarns. For example, the hm-biopolymer can be combined with natural fibers such as wool, flax or cotton. Alternatively, the hm-biopolymer is incorporated into a synthetic fiber such as polyester, nylon, rayon, acrylic, polyolefin, and spandex. In some embodiments, the hm-biopolymer (e.g., hm-chitosan) is spun into a fiber. In still other embodiments, the hm-polymer (e.g., hm-chitosan) is incorporated into the textile as flakes or particles.

Methods of making fibers and other materials based on hm-modified biopolymers can optionally be based on known processes, such as those described in one or more of U.S. Pat. Nos. 8,899,277, 9,226,988, 8,722,081, and US 2014/0242870, the entire contents of which are hereby incorporated by reference.

In some embodiments, the hm-polymer is formed from a dehydrated solution or foam, which has the potential to alter characteristics such as flexibility and feel of the resulting fabric.

Textiles in accordance with the disclosure include athletic wear, work wear, footwear, headwear, outerwear, undergarments, and medical textiles (including wound dressings) and hospital apparel, among others.

In some embodiments, hm-chitosan or hm-chitosan material is used as a dressing for skin grafts or surgical wounds (e.g., in the case of cosmetic surgery), to decrease the microbial burden on the wound site, and decrease the likelihood of MRSA or other infection. In some embodiments, hm-chitosan is applied as a gel, foam, or cream (as an alternative or in addition to its inclusion in the wound dressing). Hm-chitosan may be used in contact with the wound continually through the healing cycle, for example, for at least about 1 week, or at least about 2 weeks, or at least about 1 month, or more. In some embodiments, the composition (either wound dressing or topical composition is applied to an existing MRSA infection.

In some embodiments, hm-chitosan (as textile, or as topical foam or ointment) may also provide synergistic benefits with topical or systemic antibiotic therapy to combat existing or chronic infections, including MRSA or other bacteria that exhibits some resistance to antibiotic therapy. In various embodiments, the antibiotic can be a beta-lactam antibiotic, macrolide, or tetracycline. Exemplary antibiotics include clindamycin, erythromycin, tetracycline, minocycline, doxycycline, oxytetracycline, or lymecycline. Alternatively, the antibiotic may be selected from benzylpenicillin, amoxicillin, ampicillin, dicloxacillin, methicillin, nafcillin, oxacillin, penicillin G, cephalexin, cefoxitin, cephalolothin, ceftriaxone, ciprofloxacin, chloramphenicol, vancomycin, fusidic acid, moxifloxacin, linezolid, rifampicin, ertapenem, taurolidine, or a combination thereof. In some embodiments, a beta-lactam antibiotic (such as amoxicillin) is administered with a beta-lactamase inhibitor (e.g., clavulanate).

Other aspects and embodiments of the invention will be apparent to the skilled artisan from this disclosure.

1. A textile material comprising a hydrophobically-modified biopolymer.

2. The textile material of claim 1, wherein hydrophobically-modified biopolymer has antimicrobial properties.

3. The textile material of claim 2, wherein the hydrophobically-modified biopolymer has antimicrobial properties against one or more of: Pseudomonas aeruginosa, Acinetobacter baumanni, Klebsiella pneumonia, Escherichia coli, Staphylococcus aureus and Enterococcus faecalis.

4. The textile material of claim 3, wherein the hydrophobically-modified biopolymer has antimicrobial properties against MRSA.

5. The textile material of any one of claims 1 to 4, wherein the hydrophobically-modified biopolymer has antimicrobial properties greater than native chitosan.

6. The textile material of claim 5, wherein the hydrophobically-modified biopolymer is hydrophobically-modified chitosan.

7. The textile material of claim 6, wherein the hydrophobic moieties are selected for the antimicrobial activity, durability, flexibility, and/or water repellant nature of the modified biopolymer

8. The textile material of claim 6, wherein the chitosan is modified with hydrophobic groups having from 4 to 100 carbon atoms.

9. The textile material of claim 6, wherein the chitosan is modified with hydrophobic moieties that are linear or branched alkanes.

10. The textile material of claim 6, wherein the chitosan is modified with carbocyclic or heterocyclic hydrophobic moieties.

11. The method of any one of claims 6 to 10, wherein the hydrophobic moieties are covalently attached to as many as 50% of available amines of the polymer backbone.

12. The method of any one of claims 1 to 11, wherein the hydrophobically-modified biopolymer is incorporated into a natural or synthetic fiber.

13. The method of claim 12, wherein the fiber is wool, flax or cotton.

14. The method of claim 12, wherein the synthetic fiber is one or more of: polyester, nylon, rayon, acrylic, polyolefin, and spandex.

15. The method of any one of claims 1 to 11, wherein the hydrophobically-modified chitosan is spun into a fiber.

16. The method of any one of claims 1 to 11, wherein the hydrophobically-modified chitosan is incorporated into the textile as flakes or particles.

17. The method of any one of claims 1 to 11, wherein the hydrophobically-modified chitosan is a dehydrated solution or foam.

18. A method for making a textile of any one of claims 1 to 17, comprising incorporating a hydrophobically-modified biopolymer into the textile.

19. A method for treating a wound, comprising applying a hydrophobically modified polymer to the wound as a textile or topical composition, optionally with systemic or topical antibiotic treatment.

20. The method of claim 19, wherein the wound is a skin graft or surgical wound.

21. The method of claim 20, wherein the wound is a cosmetic surgery wound.
